(19) United States
(12) Patent Application Publication
(10) Pub. No.: US 2022/0331031 A1
(43) Pub. Date: Oct. 20, 2022

(54) ROBOTIC SURGICAL INTERVENTION DEVICE WITH AN ARTICULATED ARM CARRYING AN INSTRUMENT

(71) Applicant: COLLIN, BAGNEUX (FR)

(72) Inventors: Stéphane MAZALAIGUE, BAGNEUX (FR); Armand CZAPLINSKI, BAGNEUX (FR)

(21) Appl. No.: 17/763,066

(22) PCT Filed: Sep. 21, 2020
(86) PCT No.: PCT/FR2020/051636

§ 371 Date: Apr. 11, 2022

(30) Foreign Application Priority Data
Sep. 24, 2019 (FR) FR1910553

(51) Int. Cl. A61B 34/37; A61B 90/50

(52) U.S. Cl. CPC A61B 34/37; A61B 90/50; A61B 2034/742

(57) ABSTRACT

A robotic surgical intervention device includes an articulated arm with actuating motors, a surgical instrument carried by the articulated arm, a control peripheral of the articulated arm for moving a functional distal end of the surgical instrument, and a processor configured to process movement instructions provided by the control peripheral to convert them into individual control instructions for each of the actuating motors of the articulated arm. The processor includes an electronic restriction designed to add further processing to the movement instructions provided by the control peripheral that blocks any movement of the functional distal end of the surgical instrument according to at least one degree of freedom in translation or rotation predefined as prohibited along or about at least one axis of a local Cartesian coordinate system linked to the surgical instrument.

The present invention relates to a robotic device for surgical intervention, particularly in the field of otorhinolaryngology but not only.

It applies more specifically to a robotic device comprising:
an articulated arm with actuating motors;
a surgical instrument carried by the articulated arm, having a proximal end for attachment to the articulated arm and a functional distal end;
an articulated arm control peripheral for moving the functional distal end of the surgical instrument; and
means for processing movement instructions supplied by the control peripheral to convert them into individual instructions for controlling each of the actuating motors for operating the articulated arm.

Such a device is described in the paper by Miroir et al, entitled “RobOtol: from design to evaluation of a robot for middle ear surgery”, published at the IEEE/RSJ International Conference on Intelligent Robots and Systems held on 18-22 Oct. 2010 in Taipei, Taiwan. It presents an architecture and kinematics particularly well suited to otological surgical interventions on the middle or inner ear of patients. These interventions are sensitive to wrong movements so that robotic assistance is a valuable aid.

Nevertheless, even with this assistance, the practitioner may make a wrong gesture when manipulating the control peripheral, given the confined volume in which he or she must generally operate. If in most cases, the precision of the surgical gesture is such that a slight deviation is of no consequence and can easily be corrected, there are particular situations in which the tolerance is zero or almost zero. This is the case, for example, when operating a linear engagement or disengagement of an otological surgical instrument inside a patient's ear, or to clear a visualization axis, for example the optical axis of a microscope, without moving the functional distal end of the instrument.

More generally, in any type of surgical intervention assisted by a robotic device carrying a surgical instrument and manipulated with the help of a control peripheral, the situations in which the slightest inaccuracy can have serious consequences are numerous.

It may thus be desired to provide a robotic device that avoids at least some of the abovementioned problems and constraints.

It is therefore proposed to provide a robotic surgical intervention device comprising:
an articulated arm with actuating motors;
a surgical instrument carried by the articulated arm, having a proximal end for attachment to the articulated arm and a functional distal end;
a control peripheral of the articulated arm for moving the functional distal end of the surgical instrument; and
means for processing movement instructions supplied by the control peripheral to convert them into individual control instructions for controlling each of the actuating motors for operating the articulated arm.
   wherein the processing means include an electronic restriction designed to add further processing to the movement instructions provided by the control device consisting of blocking any movement of the functional distal end of the surgical instrument according to at least one degree of translational or rotational freedom predefined as prohibited along or about at least one axis of a local Cartesian coordinate system linked to the surgical instrument.

Thus, the electronic restriction works as a filter for predefined undesired movements with respect to axes linked to the surgical instrument. In the above-mentioned tricky situations, this prevents deviation from the desired sensitive movements regardless of the instructions issued by the control peripheral. For example, in otology, for precise linear engagement or disengagement of a surgical instrument within a patient's ear, it is sufficient for the electronic restriction to be designed in accordance with the present invention to block any movement of the functional distal end of the surgical instrument in translational and rotational degrees of freedom along and about the two axes of the local Cartesian frame of reference related to the surgical instrument other than the one on which the linear engagement or disengagement is desired. Similarly, in order to disengage a viewing axis without damage, it is sufficient for the software restriction to be programmed in accordance with the present invention to block any translation along the three axes of the local Cartesian coordinate system linked to the surgical instrument. More generally, and depending on the precise gestures desired, it is advantageous to be able to prohibit, thanks to the present invention, certain translational or rotational movements along or around at least one axis of a local Cartesian coordinate system linked to the surgical instrument.

Optionally, the control device is a 6D joystick.

Also optionally, a robotic surgical intervention device according to the invention may comprise means for activating and deactivating the electronic restriction.

Also optionally, the electronic restriction comprises a blocking of any movement outside of a translational degree of freedom and a rotational degree of freedom along and around a main axis of the surgical instrument forming a first axis around which the local Cartesian coordinate system is defined.

Also optionally, the main axis of the surgical instrument is that which connects a central point of its proximal attachment end to a central point of its functional distal end.

Also optionally and alternatively, the main axis of the surgical instrument is that of a straight distal portion of thereof, offset from the axis that connects a central point of its proximal attachment end to a central point of the functional distal end of its straight distal portion.

Also optionally:
the instructions provided by the control peripheral are expressed in a global coordinate system linked to a fixed base of the robotic device;
the processing means includes a Jacobian converter of the instructions expressed in this global coordinate system into individual instructions for controlling each of the actuating motors of the articulated arm using Jacobian parameters stored in memory; and
the electronic restriction is programmed to:
   convert the instructions provided by the control peripheral into local movement instructions expressed in the local Cartesian coordinate system of the surgical instrument,
   delete any component of those local movement instructions relating to the said at least one prohibited translational or rotational degree of freedom, to provide restricted local movement instructions,
   convert the restricted local movement instructions into restricted movement instructions expressed in the global coordinate system, and
   provide the restricted movement instructions expressed in the global coordinate system to the Jacobian converter.

Also optionally, the electronic restriction includes a blocking of any translation of the functional distal end of the surgical instrument in its local coordinate system.

Also optionally:
the articulated arm has, from its base to its carrying end, three motorized prismatic links in series followed by three motorized rotoid links in series, the three respective axes of rotation of the three rotoid links converging at a single central point of the functional distal end of the surgical instrument;
the instructions provided by the control peripheral are expressed in a global coordinate system linked to a fixed base of the robotic device;
the processing means include a Jacobian converter of the instructions expressed in this global coordinate system into individual instructions for controlling each of the actuating motors of the articulated arm using Jacobian parameters stored in memory; and
the electronic restriction is designed so as to, after application of the Jacobian converter, delete the individual control instructions of the actuating motors of the three prismatic links.

Also optionally, a robotic surgical intervention device according to the invention may be configured and dimensioned for a middle or inner ear surgical intervention of a patient, the surgical instrument itself being a patient's middle or inner ear surgical intervention instrument.

The invention will be better understood with the aid of the following description, which is given solely by way of example and is made with reference to the appended drawings wherein:

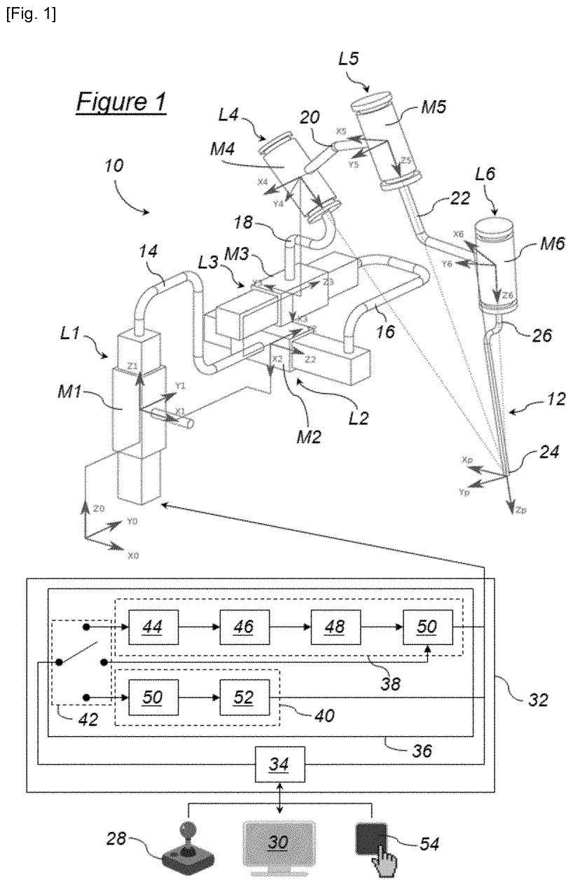

FIG. 1 diagrammatically represents the general structure of a robotic surgical intervention device, according to an embodiment of the invention,

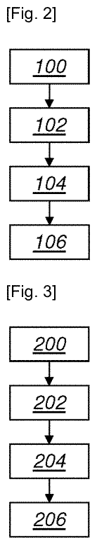

FIG. 2 illustrates the successive steps of a surgical intervention method using the robotic device of FIG. 1, according to a first embodiment of the invention, and

FIG. 3 illustrates the successive steps of a surgical intervention method using the robotic device of FIG. 1, according to a second embodiment of the invention.

With reference to FIG. 1, a robotic surgical intervention device according to an embodiment of the invention comprises an articulated arm 10 with actuating motors carrying a surgical instrument 12. The non-limiting example illustrated in this figure is more specifically that of a robotic device for an application in otological surgery of the middle or inner ear of a patient, the architecture and kinematics of which are optimized in accordance with the teaching of the above-mentioned document by Miroir et al. The articulated arm 10 thus has, from its base to its end carrying the surgical instrument 12, three motorized prismatic links in series followed by three motorized rotoid links in series.

A first prismatic link L1, driven by a first motor M1, allows translational movement of a first member 14 of the articulated arm 10 along the axis Z1 (e.g. vertical) of a first local orthogonal Cartesian coordinate system (X1, Y1, Z1) linked to the first motor M1. The first motor M1 is attached to the robotic device so that the first local coordinate system (X1, Y1, Z1) has the same directions as a global orthogonal Cartesian coordinate system (X0, Y0, Z0) linked to a fixed base of the robotic device. The movement axis of the first member 14 is therefore parallel to Z0.

A second prismatic link L2, actuated by a second motor M2 carried by one end of the first member 14, allows the translational displacement of a second member 16 of the articulated arm 10, along the axis Z2 of a second local orthogonal Cartesian coordinate system (X2, Y2, Z2) linked to the second motor M2. The second local coordinate system (X2, Y2, Z2) is turned through a right angle with respect to the Y1 axis of the first local coordinate system (X1, Y1, Z1) so that its Z2 axis is parallel to the X1 axis. The movement axis of the second member 16 is therefore parallel to X0.

A third prismatic link L3, actuated by a third motor M3 carried by one end of the second member 16, allows the translational displacement of a third member 18 of the articulated arm 10, along the axis Z3 of a third local orthogonal Cartesian coordinate system (X3, Y3, Z3) linked to the third motor M3. The third local coordinate system (X3, Y3, Z3) is turned through a right angle with respect to the X2 axis of the second local coordinate system (X2, Y2, Z2) so that its Z3 axis is parallel to the Y2 axis which is itself parallel to the Y1 axis. The movement axis of the third member 18 is therefore parallel to Y0.

A fourth rotoid link L4, actuated by a fourth cylindrical motor M4 and carried by one end of the third member 18, allows the rotational movement of a fourth member 20 of the articulated arm 10, about the axis Z4 of a fourth local orthogonal Cartesian coordinate system (X4, Y4, Z4) linked to the fourth motor M4.

A fifth rotoid link L5, actuated by a fifth cylindrical motor M5 and carried by one end of the fourth member 20, allows the rotational movement of a fifth member 22 of the articulated arm 10, about the axis Z5 of a fifth local orthogonal Cartesian coordinate system (X5, Y5, Z5) linked to the fifth motor M5.

Finally, a sixth rotoid link L6, actuated by a sixth cylindrical motor M6 and carried by one end of the fifth member 22, allows the rotational movement of the surgical instrument 12, about the axis Z6 of a sixth local orthogonal Cartesian coordinate system (X6, Y6, Z6) linked to the sixth motor M6.

According to the particularly interesting configuration of FIG. 1, the three respective rotation axes Z4, Z5 and Z6 of the three rotoid links converge at a same central point of the functional distal end 24 of the surgical instrument 12, thus making this point a pivot point. This means that in the absence of any actuation of the motors M1, M2, M3 of the prismatic links, any instruction to actuate at least one of the motors M4, M5, M6 of the rotoid links causes the surgical instrument 12 to rotate about its pivot point without any movement of the latter in the global coordinate system (X0, Y0, Z0).

The surgical instrument 12 has a proximal end 26 for attachment to the articulated arm 10, more precisely to a corresponding attachment end of the arm 10 linked to the motor M6. This attachment is for example advantageously made in accordance with the locking system described in patent FR 2 998 344 B1, but this is not mandatory. Any other fastening system suitable for the intended application is also suitable.

The surgical instrument 12 may have a rectilinear shape such that its main axis Zp, about which a local Cartesian coordinate system (Xp, Yp, Zp) is defined and linked to it, is that which connects a central point of its proximal attachment end 26 to the pivot point of its distal functional end 24. In this case, not shown in FIG. 1, the Zp axis merges with the Z6 axis.

Alternatively, and as illustrated in FIG. 1, it may be a surgical instrument with deviated portions such as that described in patent application FR 3 066 378 A1. In this case, its main axis Zp, around which the local Cartesian coordinate system (Xp, Yp, Zp) linked to it is still defined, is that of a rectilinear distal portion of this instrument, off-axis with respect to the axis Z6 which still connects the central point of its proximal fixing end 26 to the pivot point of its functional distal end 24.

The robotic surgical intervention device further comprises a peripheral control device 28 for the articulated arm 10, such as a 6D joystick or any other equivalent device, adapted to allow a movement of the functional distal end 24 of the surgical instrument 12 according to three degrees of freedom in translation and three degrees of freedom in rotation by actuating the six motors M1 to M6. It may also include a screen 30, in particular for displaying and monitoring any movement of the surgical instrument 12 during the operating phase.

The robotic surgical intervention device further comprises means for processing movement instructions provided by the control peripheral 28 to convert them into individual instructions for controlling each of the motors M1 to M6 of the articulated arm 10. These processing means take the form of an electronic circuit 32.

The electronic circuit 32 is connected to the articulated arm 10 in order to transmit thereto the individual instructions for controlling the motors M1 to M6 and to the control peripheral 28 in order to receive its movement instructions. These instructions are generally expressed in the global coordinate system (X0, Y0, Z0).

It has a central processing unit 34, such as a microprocessor designed to send the individual control instructions to the articulated arm 10 and to receive movement instructions from the control peripheral 28, and a memory 36 in which at least one computer program performing the aforementioned conversion and intended to be executed by the central unit 34 is stored. Two computer programs 38 and 40, selectable according to a software switch 42, are shown in FIG. 1. They relate to two different applications of the present invention which implement two functionally different but possibly complementary embodiments. Only one of the two programs could be implemented without going beyond the scope of the present invention.

In accordance with one possible embodiment of the present invention, each of the two computer programs 38, 40 includes instructions for implementing a software restriction programmed to add further processing to the movement instructions provided by the control peripheral 28 consisting of blocking any movement of the functional distal end 24 of the surgical instrument 12 according to at least one translational or rotational degree of freedom predefined as prohibited along or about at least one axis of the local coordinate system (Xp, Yp, Zp).

It should be noted that the electronic circuit 32 as schematically represented in FIG. 1 may, for example, be implemented in a computer device such as a conventional computer comprising a processor associated with one or more memories for the storage of data files and computer programs whose instructions are intended to be executed by the processor, such as the instructions of the programs 38, 40 and of the software switch 42 which may also constitute a computer program. These programs are shown as separate, but this distinction is purely functional. They could just as easily be grouped according to any combination into one or more software programs. Their functions could also be at least partly micro-programmed or micro-wired into dedicated integrated circuits. Thus, alternatively, the computer device implementing the electronic circuit 32 could be replaced by an electronic device made of digital circuits only (without a computer program) for performing the same actions. In particular, the aforementioned software restriction is more generally an electronic restriction whose function can be implemented in hardware and/or software.

In addition to the electronic restriction implemented in the electronic circuit 32, the robotic surgical intervention device is optionally but advantageously provided with means 54 for activating and deactivating this electronic restriction. Any existing selection device is conceivable, in particular a touch or mouse selectable button of the display screen 30, a specific device of the control peripheral 28, or even an independent selection device provided in another peripheral, for example on the keyboard or by means of a pedal.

In the example shown in FIG. 1, as discussed above, the electronic restriction actually includes two different functional restrictions which are particularly convenient and clever in otologic surgery. A first functional restriction is programmed in the computer program 38. It is designed to block any movement outside of one translational degree of freedom and one rotational degree of freedom along and around the main axis Zp of the surgical instrument 12 regardless of its straight or deviated shape. It corresponds to the possibility of operating in otology, simply and quickly in an intuitive way, a precise linear engagement or disengagement of the surgical instrument 12 inside a patient's ear without any wrong gesture. A second functional restriction is programmed in the computer program 40. It is designed to block any translation along the three axes Xp, Yp and Zp of the local coordinate system linked to the surgical instrument 12. It corresponds to the possibility of operating in otology, simply and quickly in an intuitive way, to release without damage a visualization axis of the functional distal end 24 of the surgical instrument 12 towards the patient's ear, for example an optical axis of a microscope whose use is notably envisaged in the robotic device of the aforementioned document Miroir et al. Other functional restrictions can more generally be envisaged according to the precise gestures desired and notably programmed in the computer program 38. They all consist of prohibiting certain translational or rotational movements along or around at least one axis of the local coordinate system (Xp, Yp, Zp) linked to the surgical instrument 12. All these possible electronic restrictions are advantageously activated during tricky interventions in confined cone-shaped volumes such as those of the middle or inner ear of a patient.

More precisely, the computer program 38 includes instructions 44 for performing a conversion of the instructions provided by the control peripheral 28, expressed in the global coordinate system (X0, Y0, Z0), into local movement instructions expressed in the local coordinate system (Xp, Yp, Zp) of the surgical instrument 12. A simple knowledge of the layout of the surgical instrument 12 in space makes it possible to easily reconstitute the transfer matrix performing this conversion.

The computer program 38 includes instructions 46, to be executed after the instructions 44, for implementing the first functional restriction, i.e., a deletion of the components of these local movement instructions along and about the Xp and Yp axes to keep only the translational and rotational degrees of freedom along and about the principal axis Zp of the surgical instrument 12. It should be noted that these instructions can be generalized to implement other functional restrictions with at least one prohibited translational or rotational degree of freedom. This results in restricted local movement instructions.

The computer program 38 includes instructions 48, to be executed after the instructions 46, for performing a reverse conversion of the restricted local movement instructions expressed in the local coordinate system (Xp, Yp, Zp) into restricted movement instructions expressed in the global coordinate system (X0, Y0, Z0).

Finally, the computer program 38 includes instructions 50, intended to be executed after instructions 48, or directly without executing instructions 44, 46 and 48 if no electronic restriction is selected, for performing a Jacobian conversion of the restricted movement instructions (or unrestricted if no electronic restriction is selected) expressed in the global coordinate system (X0, Y0, Z0) into individual instructions for controlling each of the motors M1 to M6 for actuating the articulated arm 10 using Jacobian parameters stored in memory. This Jacobian converter function is well known to those skilled in the art and will not be detailed. The individual control instructions provided by execution of the computer program 38 are to be transmitted by the central unit 34 to the articulated arm 10.

The second functional restriction defined above could also be implemented by running the computer program 38, as well as other possible functional restrictions, by adapting the instructions 46. However, the program 40 takes advantage of the particular architecture and kinematics of the articulated arm 10 of FIG. 1 to simplify its implementation. In fact, in order to implement this second functional restriction and as seen previously, it is sufficient to delete the individual instructions intended for the motors M1, M2 and M3 and resulting from the aforementioned Jacobian conversion.

The computer program 40 thus includes the aforementioned instructions 50 for directly performing the Jacobian conversion of control instructions provided by the control peripheral 28.

It further includes instructions 52, intended to be executed after the instructions 50, for performing the second functional restriction, i.e. a deletion of the individual control instructions of the motors M1, M2 and M3 for actuating the three prismatic links L1, L2 and L3 respectively. The individual control instructions provided by execution of the computer program 40 are to be transmitted by the central unit 34 to the articulated arm 10.

The software switch 42 allows the selection of the execution of the entire computer program 38, the instructions 50 of the computer program 38 only, or the computer program 40, depending on whether or not either of the first and second electronic restrictions is selected by the activating and deactivating means 54.

FIG. 2 illustrates the successive steps of a surgical intervention method using the robotic device of FIG. 1, according to a first embodiment of the invention consisting of applying the first electronic restriction.

At a first step 100, the first electronic restriction is activated and an operator initiates the movement of the functional distal end 24 of the surgical instrument 12 carried by the articulated arm 10 using the control peripheral 28.

At a subsequent step 102, the central processing unit 34 executes instructions 44, 46, 48 and 50 of the computer program 38 to convert the instructions provided by the control peripheral 28 into individual restricted instructions for controlling the motors M1 to M6. These restricted instructions allow only translational or rotational movement along or about the Zp axis of the functional distal end 24 of the surgical instrument 12 so that, regardless of any deviations introduced by manipulation of the control peripheral 28, only the desired linear engagement or disengagement is executed quickly and intuitively during this step.

At a subsequent step 104, the first electronic restriction is deactivated. This makes it possible, for example at a subsequent step 106, to initiate any further movement of the surgical instrument 12, according to all degrees of freedom allowed by free actuation of the motors M1 to M6, by execution only of the instructions 50 of the computer program 38 instructions 50. At any time, a return to step 100 is possible.

FIG. 3 illustrates the successive steps of a surgical intervention method using the robotic device of FIG. 1, according to a second embodiment of the invention consisting of applying the second electronic restriction.

At a first step 200, the second electronic restriction is activated and an operator initiates a movement of the surgical instrument 12 carried by the articulated arm 10 using the control peripheral 28, to clear a viewing axis towards the patient's ear.

At a subsequent step 202, the central processing unit 34 executes instructions 50 and 52 of the computer program 40 to convert the instructions provided by the control peripheral 28 into individual restricted instructions for controlling the motors M4 to M6. These restricted instructions allow only rotational movements about the axes Z4, Z5 and Z6. Since these axes converge at the pivot point of the surgical instrument 12, the instrument remains stationary regardless of any deviations introduced by manipulation of the control peripheral 28, with only the desired visual release being executed quickly and intuitively during this step.

At a subsequent step 204, the second electronic restriction is deactivated. This makes it possible, for example at a subsequent step 206, any further movement of the surgical instrument 12, in all degrees of freedom allowed by free actuation of the motors M1 to M6, to be initiated by execution only of the instructions 50 of the computer program 38 (or 40). At any time, a return to step 200 is possible.

The surgical intervention methods of FIGS. 2 and 3 are readily generalizable to the implementation of other electronic restrictions than the two envisaged above.

It clearly appears that a robotic device such as the one described above allows a safe surgical intervention in some situations of restricted movements preventing any wrong movement or deviation from the desired predetermined translations or rotation.

It should also be noted that the invention is not limited to the embodiments described above.

It is advantageously applied to the architecture and kinematics of the articulated arm 10 of FIG. 1, but it can be generalized to other architectures and kinematics by adapting the corresponding conversions (i.e. coordinate system changes and Jacobian conversion).

It will more generally appear to those skilled in the art that various modifications can be made to the above-described embodiments in the light of the foregoing disclosure. In the above detailed presentation of the invention, the terms used should not be construed as limiting the invention to the embodiments set forth in the present description, but should be construed to include all equivalents the anticipation of which is within the grasp of those skilled in the art by applying their general knowledge to the implementation of the teaching just disclosed to them.

1. A robotic surgical intervention device comprising:
an articulated arm with actuating motors;
a surgical instrument carried by the articulated arm, having a proximal end for attachment to the articulated arm and a functional distal end;
a control peripheral of the articulated arm for moving the functional distal end of the surgical instrument; and
a processor configured to process movement instructions supplied by the control peripheral to convert the movement instructions into individual control instructions for each of the actuating motors of the articulated arm;
wherein the processor includes an electronic restriction designed to add further processing to the movement instructions provided by the control peripheral consisting of blocking any movement of the functional distal end of the surgical instrument according to at least one degree of translational or rotational freedom predefined as prohibited along or about at least one axis of a local Cartesian coordinate system linked to the surgical instrument.

2. The robotic surgical intervention device as claimed in claim 1, wherein the control peripheral is a 6D joystick.

3. The robotic surgical intervention device as claimed in claim 1, comprising a device configured to activate and deactivate the electronic restriction.

4. The robotic surgical intervention device as claimed in claim 1, wherein the electronic restriction comprises a blocking of any movement outside of a translational degree of freedom and a rotational degree of freedom along and around a main axis of the surgical instrument forming a first axis around which the local Cartesian coordinate system is defined.

5. The robotic surgical intervention device as claimed in claim 4, wherein the main axis of the surgical instrument is that which connects a central point of its proximal attachment end to a central point of its functional distal end.

6. The robotic surgical intervention device as claimed in claim 4, wherein the main axis of the surgical instrument is that of a straight distal portion thereof, off-axis from the axis that connects a central point of its proximal attachment end to a central point of the functional distal end of its straight distal portion.

7. The robotic surgical intervention device as claimed in claim 1, wherein:
the instructions provided by the control peripheral are expressed in a global coordinate system linked to a fixed base of the robotic device;
the processor comprises a Jacobian converter of the instructions expressed in this global coordinate system into individual instructions for controlling each of the actuating motors of the articulated arm using Jacobian parameters stored in memory; and
the electronic restriction is programmed to:
convert the instructions provided by the control peripheral into local movement instructions expressed in the local Cartesian coordinate system of the surgical instrument,
delete any component of those local movement instructions relating to the said at least one prohibited translational or rotational degree of freedom, to provide restricted local movement instructions,
convert the restricted local movement instructions into restricted movement instructions expressed in the global coordinate system, and
provide the restricted movement instructions expressed in the global coordinate system to the Jacobian converter.

8. The robotic surgical intervention device as claimed in claim 1, wherein the electronic restriction comprises a blocking of any translation of the functional distal end of the surgical instrument in its local coordinate system.

9. The robotic surgical intervention device as claimed in claim 8, wherein:
the articulated arm has, from its base to its carrying end, three motorized prismatic links in series followed by three motorized rotoid links in series, the three respective axes of rotation of the three rotoid links converging at the same central point of the functional distal end of the surgical instrument;
the instructions provided by the control peripheral are expressed in a global coordinate system linked to a fixed base of the robotic device;
the processor comprises a Jacobian converter of the instructions expressed in said global coordinate system into individual instructions for controlling each of the actuating motors of the articulated arm using Jacobian parameters stored in memory; and
the electronic restriction is designed, after application of the Jacobian converter, to delete the individual control instructions of the actuating motors of the three prismatic links.

10. The robotic surgical intervention device as claimed in claim 1, configured and dimensioned for a middle or inner ear surgical intervention of a patient, the surgical instrument itself being a patient's middle or inner ear surgical intervention instrument.
